(19) United States
(12) Patent Application Publication
(10) Pub. No.: US 2008/0073595 A1
(43) Pub. Date: Mar. 27, 2008

(54) Ultra-violet cleaning device

(76) Inventor: Devaraj Thiruppathi, Columbus, GA (US)

Correspondence Address: Devaraj Thiruppathi, 7219 Stone Creek Lane, Columbus, GA, 31909, US

(21) Appl. No.: 11/485,086

(22) Filed: Jul. 12, 2006

(51) Int. Cl. G01J 3/10

(52) U.S. Cl. 250/504 R

(57) ABSTRACT

A ultra-violet cleaning and sterilization device that emits sufficient radiation to eradicate germs, bacteria, viruses and other pathogens and microorganisms with a replaceable and disposable outer fabric which cleans the surface and a ultra-violet emitting source sterilizing the surface with an on/off switch and a safety button which activates the ultra-violet emitting source by applying pressure to the button while wiping or cleaning a surface.

### FIELD OF THE INVENTION

The invention relates generally to a ultra-violet emitting cleaning and sterilization device with a disposable cleaning material and a pressure sensitive activation mechanism.

### BACKGROUND OF THE INVENTION

Ultra-violet radiation is well known to kill germs and viruses in the radiation range of 300-200 nanometers short wavelengths. Ultra-violet lamps have been used to eradicate germs, purify air, water and other surfaces. The intensity of the radiation and duration of exposure determines the potency of the ultra-violet radiation. The shorter the wavelength the stronger and quicker the kill factor. Ultra-violet light with wavelengths shorter than 300 nanometers is extremely effective in killing microorganisms. The most effective sterilizing range of UV light is within the C bandwidth of 253.7 nm. This range is called germicidal UV bandwidth or UVC. UVC radiation is harmful to humans if there is prolonged exposure to such levels of radiation. However a reduced exposure is required for damage to the eye. Therefore safety measures are required to limit the duration and activation of the UVC emitting device.

At present ultra-violet radiation is used in medical, environmental and recreational environments, such as hospitals, dental practices and tanning beds, granted a different intensity of ultra-violet radiation and duration is used in each category to obtain the desired effect.

There are many hand held devices which emits ultra-violet radiation. U.S. Pat. No. 5,920,075 issued to Whitehead (Jul. 6, 1999) discloses a device with sufficient intensity to kill germs and bacteria in a large room with a combination unlocking mechanism. U.S. Pat. No. 4,952,369 issued to Beliloa (Aug. 28, 1990) describes a pocket size portable ultra-violet flash light device that kills germs by shining and passing the ultra-violet radiation over the surface. The flash light includes a housing and a ultra-violet light source an on/off switch and a gravity switch that limits the activation of the ultra-violet light source to the position wherein the light source is facing downward. U.S. Pat. No. 4,471,226 issued to Wisnosky, Mclaughlin and McGinn III (Sep. 11, 1984) describes a portable hand held device with a safety precaution, which is force to activate only on flat surface.

There is a need for a ultra-violet cleaning and sterilization device with a cleaning material and an ultra-violet light source with sufficient intensity to kill germs, bacteria, viruses and other pathogens and microorganisms.

### SUMMARY OF THE INVENTION

This invention relates to a sterilization and cleaning device that emits ultra-violet radiation in a range sufficient to destroy germs, bacteria, viruses, pathogens and microorganisms. The device can be used by ordinary individuals to clean and at the same time sterilizing objects such as floors, toilet seats and kitchen counter tops among other things. The device includes a housing containing a ultra-violet light source, a power source, attachment site for a cleaning material and a safety mechanism containing an on/off switch and a safety button activation to radiate areas while cleaning.

The ultraviolet cleaning and sterilization device can be utilized in the form of a mop having a long upright handle and at the bottom of the handle a cleaning head and a housing containing the ultra-violet light source. The housing containing the ultra-violet light source in located on the cleaning head allowing for easy cleaning and sterilizing the surface. The cleaning head has attachment sites where disposable cleaning materials can be attached to the cleaning head. The cleaning material can be a dry material for dusting or be wet material to clean the floor at the same time sterilizing the floor. The cleaning head is pivotally attached to the bottom of the upright handle to allow for flexible varied movement of the cleaning head. The ultra-violet light source is powered by a rechargeable power source located on the upright handle. This is for the convenience of the user whereby the user only has to plug into the outlet to charge the power source and activate the ultra-violet light. There is also a safety button which as to be pressed after the on/off switch is placed in the on position to activate the ultra-violet light so there is no accidental activation of the ultra-violet light.

The ultra-violet cleaning and sterilizing device can also be utilized in the form of a hand held cleaning device to clean kitchen countertops and the like. The hand held ultra-violet cleaning and sterilizing device has a ultra-violet light source and cleaning material incorporated in a small hand held unit. The cleaning material is removably attached to the hand held unit by a hook and loop fastener, such as a VELCRO fastener. After repeated use the cleaning material can be disposed of and replaced with a new cleaning material. The ultra-violet housing containing the ultra-violet light source is located beside the removably attached cleaning material whereby allowing easy cleaning and sterilization of the countertops and other surfaces. The power source is located in a housing above the site of attachment of the removably attached cleaning material. The power source can be battery operated or be a rechargeable unit. There is also a safety button which has to be pressed after the on/off switch is placed in the on position to activate the ultra-violet light so there is no accidental activation of the ultra-violet light.

Therefore, in accordance with present invention the ultra-violet cleaning device sterilizes with the ultra-violet light and cleans with a disposable cleaning material. These and other objects, features and advantages of the present invention will become apparent from the following detailed description. It should be understood, however, that the detailed description and the specific examples, while indicating preferred embodiments of the invention, are given by way of illustrations only, since various changes and modifications within the spirit and scope of the invention will become apparent to those skilled in the art.

### BRIEF DESCRIPTION OF THE DRAWINGS

The present invention will become more fully understood from the detailed description given herein below and the accompanying drawings, which are given by way of illustration only and are not limitative of the present invention, wherein:

FIG. 1 is a plan view of the ultra-violet cleaning and sterilization device with long handle and cleaning head;

FIG. 2 is a plan view of the ultra-violet cleaning and sterilization device with long handle and cleaning head with cleaning material;

FIG. 3 is a side cross sectional view of the ultra-violet cleaning and sterilization device with long handle and cleaning head;

FIG. 4 is an elevated view of a hand held ultra-violet cleaning and sterilization device;

FIG. 5 is a bottom view of a hand held ultra-violet cleaning and sterilization device;

FIG. 6 is a cross sectional view of a hand held ultra-violet cleaning and sterilization device;

FIG. 7 is a bottom view of the hand held ultra-violet cleaning and sterilization device without the cleaning material;

FIG. 8 is a top view of the cleaning material of the hand held ultra-violet cleaning and sterilization device:

### DETAILED DESCRIPTION OF THE PREFERRED EMBODIMENT

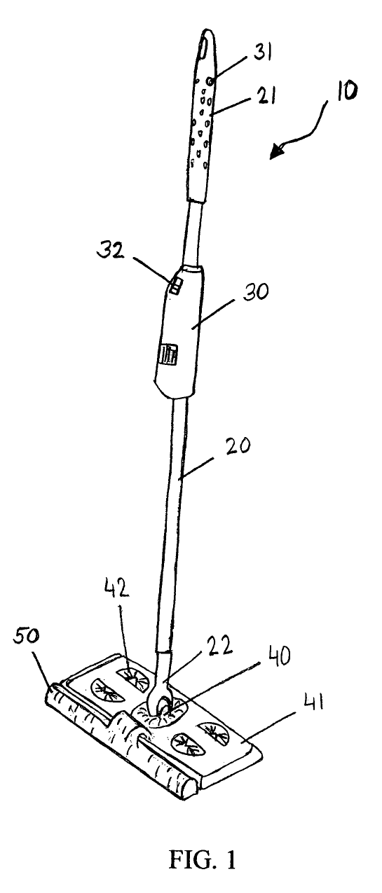

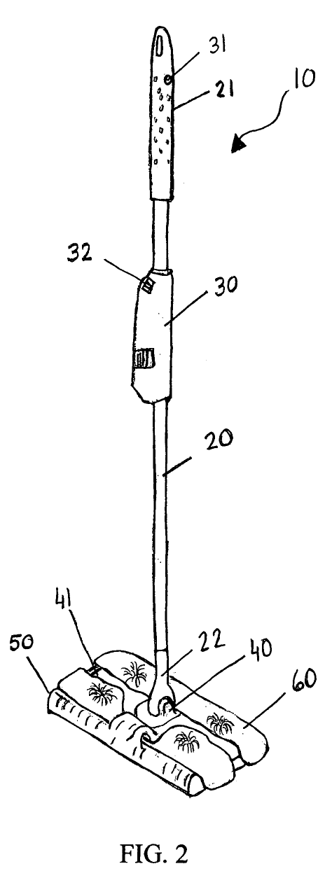

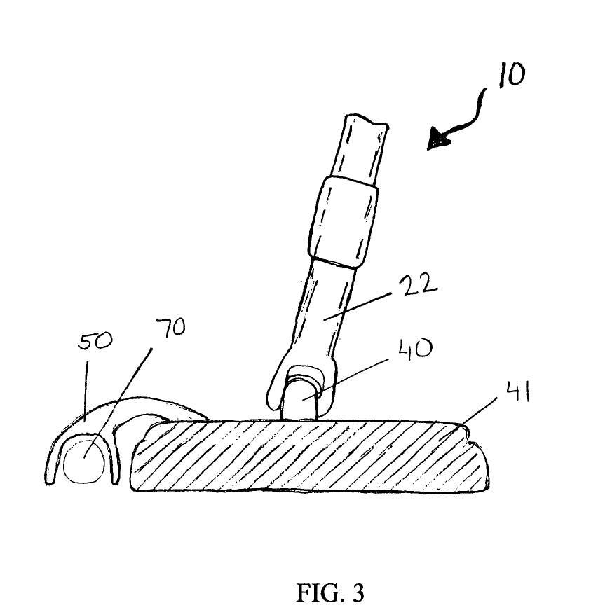

Referring now to the drawings wherein the depictions are for purposes of illustrating a preferred embodiment of the present invention only and not for the purpose of limiting the same. FIG. 1 is a plan view of the ultra-violet cleaning and sterilization device 10 of the present invention. Cleaning and sterilization device 10 is generally composed of a cleaning head 41 and a ultra-violet housing 50. The cleaning head 41 has a attachment gripper 42. FIG. 2 depicts a cleaning material 60 covers the cleaning head 41 and attaches to the attachment gripper 42 by pushing cleaning material 60 into attachment gripper 42. Ultra-violet cleaning and sterilization device 10 has a long handle 20, a rechargeable power source 30 and a top handle 21. The top handle 21 contains a safety button 31, which turns on a ultra-violet light 70 in FIG. 3. The rechargeable power source 30 has a on/off switch 32 which turns on the rechargeable power source 30. The ultra-violet light 70 only turns on if the on/off switch is in the on position and the safety button 31 is compressed. The ultra-violet light 70 is located within the ultra-violet housing 50 and sterilizes the floor while wiping the floor clean by the cleaning material 60 attached to the cleaning head 41. The cleaning material 60 maybe composed of wet or dry material cleaning the floor. FIG. 3 depicts a bottom of handle 22, which attaches to a pivoting head 40 located on the cleaning head 41 allowing for varying degree of movement of the cleaning head 41.

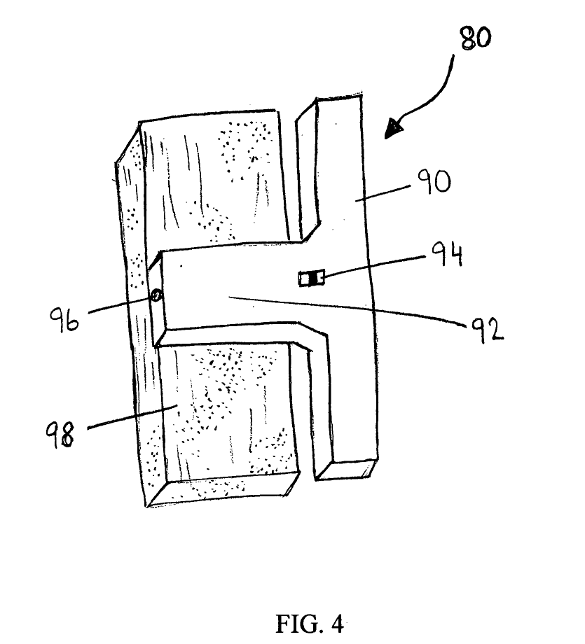

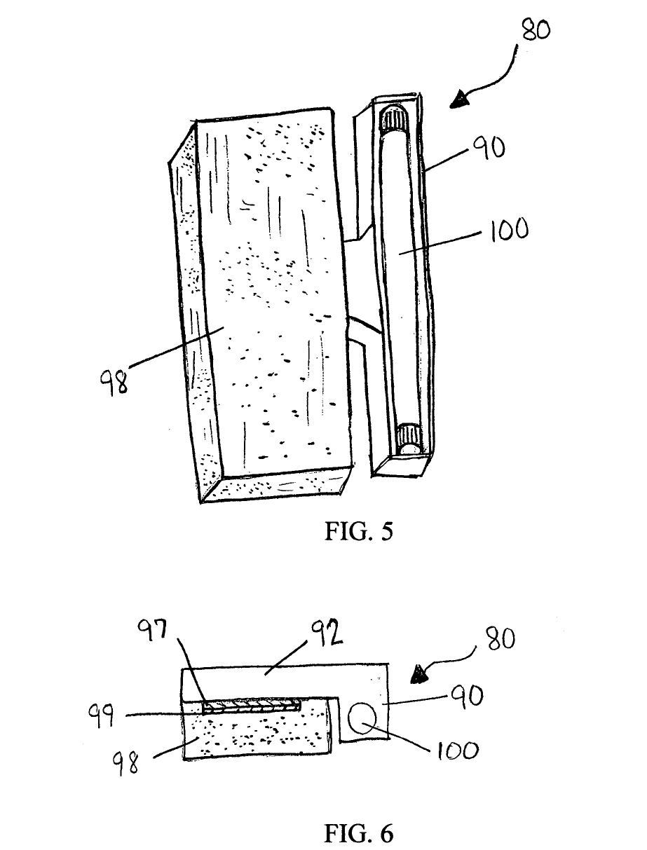

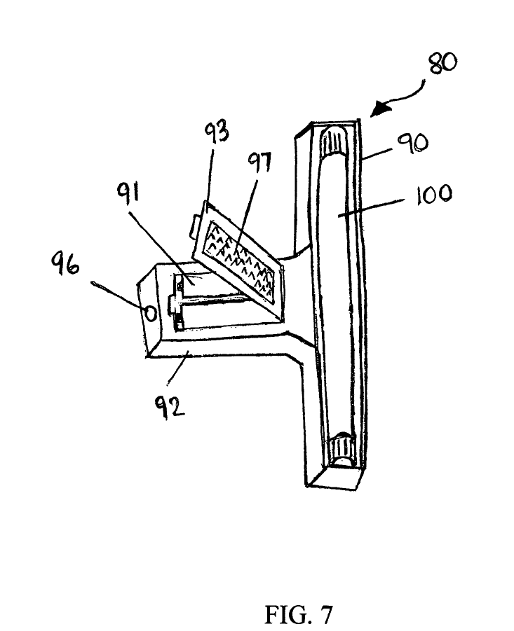

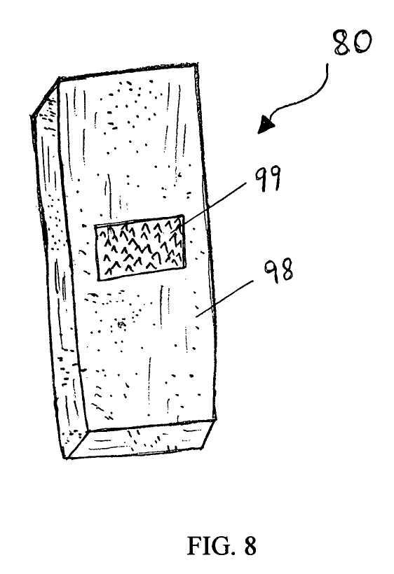

FIG. 4 is another embodiment of the ultra-violet cleaning and sterilization device. Device 80 in FIG. 4 is a hand held ultra-violet cleaning and sterilization device with a ultra-violet housing 90, a power source housing 92 and a cleaning material 98. The cleaning material 98 is removably attached to the bottom side of the power source housing 92 by a hook and loop fastener 97 and 99 as depicted in FIG. 6. FIG. 4 depicts a on/off switch 94 and a safety push button 96 to turn a ultra-violet light 100 depicted in FIG. 5 on and off. The ultra-violet light 100 only turns on when the on/off switch is in the on position and then pressing the safety push button 96. FIG. 7 depicts the bottom view of the cleaning and sterilization device without the cleaning material. A power source 91 are batteries and a battery latch 93 to secure the batteries. The hook and loop fastener 97 is located on the batter latch 93 and is removably attached to the cleaning material 98 by hook and loop fastener 99. FIG. 8 depicts the top view of cleaning material 98 with the hook and loop fastener 99. The cleaning material 98 is removably attached to the ultra-violet cleaning and sterilization device 80 and maybe of dry or wet cleaning material.

What is claimed is:

1) A portable ultra-violet cleaning and sterilization device comprising:
a) a ultra-violet light source;
b) a power source;
c) a on/off switch;
d) a cleaning material, whereby wiping clean and sterilizing the surface.

2) The portable ultra-violet cleaning and sterilization device of claim 1, wherein said cleaning material is disposable.

3) The portable ultra-violet cleaning and sterilization device of claim 2, wherein said cleaning material is a wet or dry dust cloth sheets or sponge.

4) The portable ultra-violet cleaning and sterilization device of claim 3, wherein said cleaning material is affixed by attachment means to a cleaning head which is pivotally mounted to a upright handle.

5) The portable ultra-violet cleaning and sterilization device of claim 4, wherein said ultra-violet light source is attached to said cleaning head.

6) The portable ultra-violet cleaning and sterilization device of claim 5, wherein said power source is a rechargeable battery attached to said upright handle.

7) The portable ultra-violet cleaning and sterilization device of claim 8, wherein a safety button is pressed to activate the ultra-violet light source after said no/off switch is activated.

8) The portable ultra-violet cleaning and sterilization device of claim 1, wherein a hand held ultra-violet unit incorporates said power source and said ultra-violet light source.

9) The portable ultra-violet cleaning and sterilization device of claim 8, wherein said cleaning material is attached by attachment means to said hand held ultra-violet unit.

10) The portable ultra-violet cleaning and sterilization device of claim 9, wherein said cleaning material consists of either dry or wet sheet or sponge and is disposable.

11) The portable ultra-violet cleaning and sterilization device of claim 10, wherein said power source contains said on/off switch and a safety button, whereby said safety button has to be press to activate the ultra-violet light source while said on/off switch is in the on position.

12) The portable ultra-violet cleaning and sterilization device comprising a disposable cleaning material and a housing encompassing a ultra-violet light source, wherein said housing encompassing said ultra-violet light source is mounted to a cleaning head where said cleaning material attaches by attachment means, wherein said cleaning head is pivotally attached to a upright handle wherein said upright handle contains a rechargeable power source containing a on/off switch to allow activation of said ultra-violet light source, a safety button is located on the top part of said upright handle, whereby said ultra-violet light source is activated by positioning the on/off switch to the on position and pressing said safety button.

13) The portable ultra-violet cleaning and sterilization device comprising a disposable cleaning material and a housing encompassing a ultra-violet light source, wherein said housing encompassing said ultra-violet light source is incorporated into a hand held unit where said cleaning material attaches by attachment means to said hand held unit, which has a power source, a on/off switch and a safety button whereby said ultra-violet light source is activated by positioning the on/off switch to the on position and pressing said safety button.
